(19) United States
(12) Patent Application Publication
(10) Pub. No.: US 2009/0155295 A1
(43) Pub. Date: Jun. 18, 2009

(54) VACCINE

(76) Inventors: Helen Susan Atkins, Salisbury (GB); David Neil Harland, Salisbury (GB); Stephen John Elvin, Salisbury (GB); Richard William Titball, Exeter (GB)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/096,969

(22) PCT Filed: Dec. 11, 2006
(86) PCT No.: PCT/GB2006/004627

§ 371 Date: Nov. 5, 2008

(30) Foreign Application Priority Data
Dec. 12, 2005 (GB) 0525213.5

(51) Int. Cl. A61K 39/02; C07K 14/195; C12N 15/11; A61K 31/7088; C07K 16/18

(52) U.S. Cl. 424/190.1; 530/350; 536/23.7; 514/44; 530/387.9

(57) ABSTRACT

A polypeptide derived from the periplasmic space of Yersinia pestis or a fragment or a variant of said polypeptide for use as a medicament is described. Pharmaceutical compositions comprising such polypeptides, such as OppA from Yersinia pestis, are also described and claimed. In a preferred embodiments the pharmaceutical composition further comprises existing protective antigens from Y. pestis such as F1 and V antigens. Such compositions are protective in mice and may be used as a vaccine for the prophylactic or therapeutic treatment of plague.

This invention relates to polypeptides derived from the periplasmic space of Yersinia pestis and their use in providing protection against plague. In particular, this invention relates to pharmaceutical compositions comprising Y. pestis periplasmic solute binding proteins and their use as vaccines against Y. pestis infection.

Yersinia pestis is the causative agent of plague and globally between 1,000 and 3,000 cases of plague are reported to the World Health Organization (WHO) each year. Most of these cases are the bubonic form of the disease, usually a consequence of the transmission of bacteria to humans via bites from fleas that have previously fed on infected rodents. More rarely, cases of pneumonic plague are reported which are characterized by a short incubation period of 2-3 days and a high rate of mortality, even if treated. Pneumonic plague may possess a greater threat to a population since this form of the pathogen can be transmitted from person-to-person or from animal-to-person via inhalation of contaminated air droplets. Also, recent concerns have been raised over the potential use of pathogens such as Y. pestis as biological warfare agents. It is therefore important to ensure useful and viable methods of treating such infections are available. A number of antibiotics are active towards Y. pestis but their use to treat plague is highly dependent on early intervention. This is particularly important for the treatment of pneumonic plague.

A number of vaccine candidate have been identified for plague. Killed whole Y. pestis cells have been used in various plague vaccine formulations for many years. There is evidence to show that this type of vaccine is successful in providing protection against bubonic plague but the ability of killed whole cell vaccines to provide protection against pneumonic plague has not yet been adequately demonstrated. Furthermore, killed whole cell vaccines have been associated with a high incidence of side effects.

In recent years there has been significant activity in the development of subunit vaccines which aim to provide protection against both bubonic and pneumonic plague. A wide range of cell-surface molecules have been evaluated as vaccine components including lipopolysaccharide and components of the type III secretion system (Titball, R. W., and Williamson, E. D. Expert Opin. Biol. Ther. 4, (2004) pp 965-973). Further activity in this field has identified that both the F1 and V antigens of Y. pestis can induce protective immunity and improved vaccines based on these antigens are currently being developed (Williamson, E D. J. Appl. Microbiol. 91 (2001) pp 606-608). However, since F1 antigen-negative strains of Y. pestis have been isolated and reported, it is possible that use of the F1 and V antigens alone may not provide full protection against certain Y. pestis strains. It is therefore highly desirable to obtain an additional protective antigen against Y. pestis. Such an additional antigen would ideally be protective against all strains of Y. pestis when used in isolation but could also advantageously be used in conjunction, or combination, with existing sub-unit vaccines.

It has been suggested by several researchers that ATP-binding cassette (ABC) transporter proteins could be targets for development of sub-unit vaccines against pathogenic bacteria (Garmory, H. S, and R. W. Titball. Infect. Immun. 72 (2004) pp 6757-63). The ABC transporter family belongs to the primary energy-dependent transporter group and is one of the largest transporter families responsible for diverse physiological processes including drug efflux from cancer cells and bacterial nutrient uptake across the cell envelope using the free energy of hydrolysis of ATP. It is this broad diversity of function that has led to the suggestion that ABC transporters may be involved in virulence. Although this has led to the targeting of such proteins as potential vaccine antigens, it has been found that not all ABC transporters are immunogenic and that many, if not most, of the ABC transporter proteins isolated to date are largely ineffective as vaccine antigens in the models which have been tested to date, irrespective of their ability to elicit an immune response.

The inventors have now established that certain putative ABC transporter proteins derived from Yersinia pestis are both immunogenic and protective. The proteins have been cloned, expressed, characterised and their potential to induce protective immunity against Y. pestis tested in the mouse model of infection.

Specifically these proteins are polypeptides derived from the periplasmic space of Yersinia pestis or a variant of said polypeptide which is capable of producing a protective immune response against Y. pestis, or a fragment of any of these which is capable of producing a protective immune response against Y. pestis

According to a first aspect of the invention there is provided a polypeptide derived from the periplasmic space of Yersinia pestis or a variant of said polypeptide which is capable of producing a protective immune response against Y. pestis, or a fragment of any of these which is capable of producing a protective immune response against Y. pestis, for use as a medicament.

As used herein the expression “capable of producing a protective immune response against Y. pestis” means that the substance is capable of generating a protective immune response in a host organism such as a mammal for example a human, to whom it is administered.

As used herein the term “polypeptide” means a sequence of amino acids joined together by peptide bonds. The amino acid sequence of the polypeptide is determined by the sequence of the DNA bases which encode the amino acids of the polypeptide chain. The polypeptides described herein include, but are not limited to, complete proteins.

As used herein the term “fragment” refers to any portion of the given amino acid sequence of a polypeptide which has the same activity as the complete amino acid sequence. Fragments will suitably comprise at least 5 and preferably at least 10 consecutive amino acids from the basic sequence and does include combinations of such fragments. In order to retain activity, fragments will suitably comprise at least one epitopic region. Fragments comprising epitopic regions may be fused together to form a variant.

In the context of the present invention the expression “variant” as used herein refers to sequences of amino acids which differ from the base sequence from which they are derived in that one or more amino acids within the sequence are substituted for other amino acids. Amino acid substitutions may be regarded as “conservative” where an amino is replaced with a different amino acid with broadly similar properties. “Non-conservative” substitutions are where amino acids are replaced with amino acids of a different type. Broadly speaking, fewer non-conservative substitutions will be possible without altering the biological activity of the polypeptide. Suitably variants will be greater than 79% identical, preferably at least 85% identical, more preferably at least 90% identical, and most preferably at least 95% identical to the base sequence. Variants included in the description of the present invention are intended to exclude substitutions which result in the variant having a substantially identical sequence to a genomic sequence from another organism.

Identity in this instance can be judged for example using the BLAST program (vs. 2.2.12) found at http://www.ncbi.nlm.nih.gov/BLAST/ or the algorithm of Lipman-Pearson, with Ktuple: 2, gap penalty: 4, Gap Length Penalty: 12, standard PAM scoring matrix (Lipman, D. J. and Pearson, W. R., Rapid and Sensitive Protein Similarity Searches, Science, 1985, vol. 227, 1435-1441).

In particular, the polypeptide is for use as a prophylactic or therapeutic vaccine against infection by Y. pestis. Prophylactic vaccines are particularly preferred.

Suitable polypeptides derived from the periplasmic space of Yersinia pestis include ABC transporter proteins which are capable of producing a protective immune response against Y pestis,

Examples of ABC transporter proteins include CysP (CDS No. YPO3015), LoIC (CDS No. YPO1626), OppA (CDS No. YPO2182), PiuA (CDS No. YPO0956), PotF (CDS No. YPO1331), PstS (CDS No. YPO4117), TolC(CDS No. YPO0663), UgpB (CDS No. YPO3796), YrbD (CDS No. YPO3573), YfeA (CDS No. YPO2439).

In particular, it has been found that OppA is a suitable protein for use as a medicament, as it generates an immunogenic response which is protective. Therefore, such proteins and protective variants and fragments thereof form a preferred embodiment of the invention.

According to a second aspect of the invention there is provided an pharmaceutical composition comprising a polypeptide derived from the periplasmic space of Yersinia pestis or a variant of said polypeptide which is capable of producing a protective immune response against Y, pestis, or a fragment of any of these which is capable of producing a protective immune response against Y. pestis in combination with a pharmaceutically acceptable carrier or excipient.

Suitable excipients and carriers will be known to those skilled in the art. These may include solid or liquid carriers. Suitable liquid carriers include water or saline. The polypeptides of the composition may be formulated into an emulsion or alternatively they may be formulated in, or together with, biodegradable microspheres or liposomes.

Suitably the composition further comprises an adjuvant which stimulates the host's immune response. Particularly suitable adjuvants include Alhydrogel, MPL+TDM and Freunds Incomplete Adjuvant.

In a preferred embodiment, the composition further comprises an additional immunogenic polypeptide derived from Yersinia pestis. The additional polypeptide may be another of those selected from the above group of polypeptides, but preferably the additional immunogenic polypeptide will be a known protective antigen for Yersinia pestis, such as the F1 antigen or the V antigen. It will be understood by the person skilled in the art that more than one additional immunogenic polypeptide can be used in combination and examples of such combinations include using the F1 and V antigens together and using a fusion protein comprising both the F1 and V antigens. Such antigens may be used in the form described in WO 96/28511, the contents of which are incorporated herein by reference.

In accordance with a third aspect of the invention, there is provided a nucleic acid which encodes a polypeptide as described above.

Such nucleic acids can be used to produce the polypeptide described above, for use in medicaments. For instance they can be incorporated into an expression vector, which is used to transform an expression host such as a prokaryotic or eukaryotic cell, and in particular is a prokaryotic cell such as E. coli using recombinant DNA technology as would be understood in the art. Such vectors, cells and expression methods form further aspects of the invention.

Alternatively, the nucleic acids can be used in “live” or “DNA” vaccines to deliver the polypeptide to the host animal.

Thus in a further aspect the invention provides a nucleic acid as described above for use as a medicament.

Particular examples of such nucleic acids are those which encode a protein of SEQ ID NO 2 hereinafter, an particular example of which is SEQ ID NO 1.

When used in this way, the nucleic acids will suitably be included into an expression vector such as a plasmid, and incorporated into a pharmaceutical composition.

Thus yet a further aspect comprises a pharmaceutical composition comprising a nucleic acid as described above in combination with a pharmaceutically acceptable carrier.

Other carriers that are suitable for use in the immunogenic composition include vectors for the delivery of the polypeptide, such as viral vectors such as vaccinia or adenovirus bacterial vectors or plasmids. These vectors will allow expression of the polypeptide encoded by the nucleic acid within the host animal.

Suitable viral or bacterial vectors advantageously comprise human or animal gut colonizing organisms that have been transformed using recombinant DNA to enable them to express the polypeptide or a protective epitopic part of the polypeptide. Salmonella-based vectors are particularly suitable but other live vectors are known in the art. Alternatively the composition may include so called naked DNA vaccines, wherein the nucleic acid such as DNA which encodes the required polypeptide is included in a plasmid.—

The composition of the present invention may be used as vaccine for plague. The vaccine may be administered prophylactically to those at risk of exposure to Y. pestis or may be administered as a therapeutic treatment to person who have already been exposed to Y. pestis.

The route of administration of the vaccine may be varied depending on the formulation of the polypeptides of the composition. The composition may be suited to parenteral administration (including intramuscular, subcutaneous, intradermal, intraperitoneal and intravenous administration) but may also be formulated for non-parenteral administration (including intranasal, inhalation, oral, buccal, epidermal, transcutaneous, ocular-topical, vaginal, rectal administration).

According to a further aspect the present invention relates to an antibody raised against the polypeptides described above, or a binding fragment thereof. Since the polypeptides are immunogenic, they are capable of inducing an immune response in a mammal to which they are administered. Antibodies may be raised in-vivo against the complete polypeptides, or they may be raised against suitable epitopic fragments of the polypeptides using conventional methods.

Binding fragments include Fab, F(ab′)₂, Fc and Fc′.

Antibodies may be polyclonal or monoclonal. Hybridoma cell lines which generate monoclonal antibodies of this type form a further aspect of the invention.

Antibodies themselves, for example in the form of sera comprising such antibodies may be useful in the passive vaccination and/or treatment of compromised individuals.

According to a yet further aspect of the invention there is provided a method of protecting a human or animal body from the effects of infection with Yersinia pestis comprising administering to the body a vaccine comprising a polypeptide or a pharmaceutical composition as described above. The polypeptide protein is capable of inducing a protective immune response in a mammal to which it is administered and the ability to elicit an effective immune response may be provided by an epitopic fragment or a variant of said protein. Particular examples of suitable proteins include, periplasmic solute binding proteins derived from Y. pestis or variants of these proteins. It is preferred that the protein is OppA (SEQ ID no. 2) protective fragment or a protective variant thereof.

The polypeptide may be administered to the body by means of a “live” or DNA vaccine as described above.

The polypeptide utilised in the above method (and also present in the composition as described above) may be isolated from a suitable subspecies of Yersinia pestis, such as Yersinia pestis CO92 or GB or alternatively it may be expressed in recombinant form and purified as appropriate as described as described in the examples herein.

In a preferred embodiment, an additional Y. pestis antigen is also administered as described above. Suitable additional protective antigens include, but are not limited to, the F1 antigen of Yersinia pestis or the V antigen of Yersinia pestis or protective variants or protective fragments of any of these. In an alternative embodiment, the F1 and V antigens may be used in combination or may be used in the form of a fusion protein comprising the F1 and V antigens of Yersinia pestis. Such antigens are described in WO 96/28551. The additional protective antigen used in this embodiment may comprise isolated and/or purified recombinant F1 and V antigens.

As described above, polypeptides derived from the periplasmic space of Y. pestis can produce a protective immune response. However, polypeptides of this type which are not protective alone, may enhance the effects of vaccines, in particular against Y. pestis, for example the vaccines described in WO96/28511.

A method for enhancing the effects of a vaccine against Y. pestis which comprises co-administering the vaccine with an immunogenic polypeptide derived from the periplasmic space of Y. pestis, may also form part of the invention.

Suitable polypeptides derived from the periplasmic space of Yersinia pestis include ABC transporter proteins which are capable of producing a protective immune response against Y. pestis, such as CysP (CDS No. YPO3015), OppA (CDS No. YPO2182), PiuA (CDS No. YPO0956), PotF (CDS No. YPO1331), PstS (CDS No. YPO4117), UgpB (CDS No. YPO3796), YrbD (CDS No. YPO3573) and YfeA (CDS No. YPO2439).

Alternatively, the vaccine could be co-administered with an immunogenic polypeptide derived from other Yersinia pestis ABC transporter proteins such as the inner membrane protein LoIC (CDS No. YPO1626) and the outer membrane protein ToIC (CDS No. YPO0663).

The present invention will now be described by way of example with reference to the following drawings, in which:

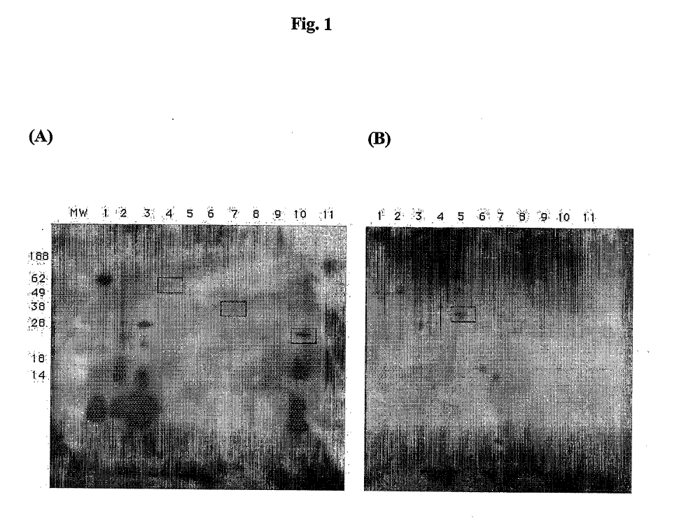

FIG. 1 shows the results obtained from Western blotting analysis of Y. pestis antigen candidate proteins. The proteins were probed with sera from (A) rabbits injected with killed Y. pestis cells or (B) humans convalescent from Y. pestis infection. The lanes are labeled as follows: molecular size indicated on figure (1) F1 antigen, (2) truncated CysP, (3) truncated LoIC, (4) full-length OppA, (5) truncated PiuA, (6) full-length PotF, (7) full-length PstS, (8) full-length ToIC, (9) full-length UgpB, (10) truncated YrbD and (11) truncated YfeD. Boxes indicate the immunoreactive proteins.

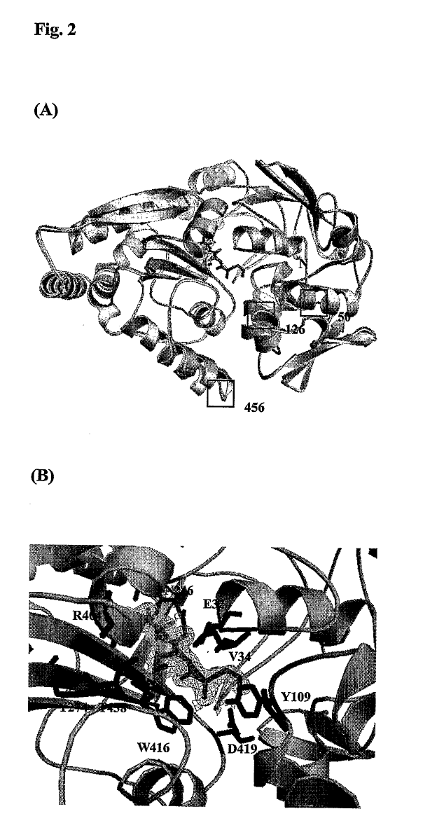

FIG. 2 shows a representation of the crystal structure of Y. pestis OppA. (A) Superimposition of the OppA structures from Y. pestis and S. typhimurium (PDB accession code 2OLB). The Lys-Lys-Lys substrate bound to the structures is shown as the small molecule in the middle of the figure (dark grey part of this molecule indicating Y. pestis and lighter grey indicating S. typhimurium). The boxes indicate the regions where the proteins show significant sequence difference. (B) Close-up view of the Lys-Lys-Lys substrate binding site of OppA from Y. pestis. The 2|Fo-F| electron density map contoured at 1.2σ is displayed on the refined structure model. The peptide backbone is also shown, and the side chains of the amino acid residues involved in substrate binding are shown in black.

### MATERIALS USED IN THE EXAMPLE

Crystallization screening kits and solutions were obtained from Hampton Research and Molecular Dimensions. Polyethylene glycol was obtained from Fluka. Antibodies were obtained from Sigma Aldrich and Serotec. All other chemicals were obtained from Sigma Aldrich and BDH Biosciences.

Identification, Cloning, Expression, and Purification of his-Tagged Antigen Candidate Proteins.

Candidate proteins (as shown in Table 1) CysP, LoIC, OppA, PiuA, PotF, PstS, ToIC, UgpB, YfeA, and YrbD were cloned, expressed and purified using the following general procedure. The Y. pestis proteins were expressed using a pET vector system and purified using Ni²⁺-NTA affinity chromatography. The purity of the proteins was ascertained by SDS-PAGE after staining with Coomassie brilliant blue R250 and the concentrations of the proteins were measured by the Bradford method (Bio-Rad) using BSA as a protein standard. The encoding genes were expressed in E. coli and the proteins isolated to 80-90% purity, a level suitable for both crystallization trials and immunological studies (Table 1).

The following procedure for the cloning, expression and purification of OppA exemplifies the procedure used for each of the protein listed in Table 1. OppA was identified in the Y. pestis CO92 genome sequence (Parkhill et al., Nature 413 (2001) pp 523-527), and oligonucleotide primers were designed to amplify the OppA-encoding gene. Briefly, the OppA gene was amplified from Y. pestis genomic DNA by PCR using the oligopeptide primers,

5′-GAATTCATGACCAACATCACAAAGAAGAATCTC-3′
and
5′-CTCGAGCTGCTTGATAATATAAAGATCTTTAACGTG-3′

then cloned into the expression vector pET24a (Novagen) using EcoRI and Xhol. The recombinant plasmid was transformed into E coli BL21 (DE3) cells (Invitrogen Ltd., Paisley, UK) and transformed cells were grown in LB medium supplemented with 50 μg/ml kanamycin at 37° C. Expression of the OppA protein was induced by 0.5 mM IPTG at OD₆₀₀=0.5, and cell cultures were harvested 4 hours after induction. E coli BL21 (DE3) cells expressing OppA were treated with 1% (w/v) lysozyme in treatment buffer (200 mM Tris-HCl pH 8.8, 20 mM EDTA-Na, 500 mM sucrose) and then the periplasmic fraction was separated from whole cells by centrifugation at 12000×g for 15 min. The supernatant was isolated and used for purification of OppA. To purify, the concentrated OppA solution was incubated with Ni²⁺ resin equilibrated with buffer A (50 mM Tris-HCl pH 7.5, 100 mM NaCl, 5 mM imidazole) for 2 h at 4° C. After extensive washing of the resin, the protein was eluted with buffer A plus 100 mM imidazole. Subsequently, the buffer was exchanged to 10 mM HEPES-NaOH pH7.5. The purity of the proteins was ascertained by SDS-PAGE after staining with Coomassie brilliant blue R250. For SDS-PAGE, the OppA protein sample was suspended in 5×times SDS sample buffer (125 mM Tri-HCl pH 6.8, 10% glycerol, 2% SDS, 25 mM DTT) and then loaded onto 12% Bis-Tris polyacrylamide gels containing 0.1% SDS. The gels were stained by Coomassie brilliant blue in 30% methanol and 10% acetic acid. The concentrations of the proteins were measured by the Bradford method (Bio-Rad) using BSA as a protein standard.

TABLE 1
Vaccine candidate proteins from Y. pestis

| | | | Amino acid |
|---|---|---|---|
| | Predicted Function | CDS number | residue number* |
| CysP | Sulfate periplasmic binding | YPO3015 | 36-345 (1-345) |
| | protein | | |
| LolC | Lipoprotein export permease | YPO1626 | 48-267 (1-400) |
| OppA | Oligopeptide periplasmic | YPO2182 | 1-545 |
| | binding protein | | |
| PiuA | Outer membrane iron | YPO0956 | 21-321 (1-699) |
| | transporter | | |
| PotF | Puristine periplasmic binding | YPO1331 | 1-369 |
| | protein | | |
| PstS | Phosphate periplasmic binding | YPO4117 | 1-346 |
| | protein | | |
| TolC | Outermembrane efflux protein | YPO0663 | 1-467 |
| UgpB | Sn glycerol 3 phosphate | YPO3796 | 1-439 |
| | periplasmic binding protein | | |
| YrbD | Organic solvent transporter | YPO3573 | 27-185 (1-185) |
| YfeA | Iron manganese periplasmic | YPO2439 | 44-323 (1-323) |
| | binding protein | | |

*The amino acid residue number refers to the portion of the protein which was expressed. In the case of the truncated proteins, the size of the complete protein is given in parentheses.

### Western Blot and ELISA Analysis of Serum Samples.

For Western blot analysis, purified Y. pestis proteins were separated in 12% SDS-PAGE gels, transferred onto PVDF membrane, and then probed with appropriate antiserum. Either rabbit antisera from animals exposed to heat-killed Y. pestis strain JAVA9 or serum from humans who had recovered from a Y. pestis infection (1:2000 dilution) was used as the primary antibody, and goat anti-rabbit or goat anti-human HRP-conjugated immunoglobulin G (IgG) (1:10,000 dilution), as appropriate, was used as the secondary antibody. F1 antigen was used as a positive control. Labeled proteins were detected using the ECL kit (Amersham Biosciences).

The purified Y. pestis proteins were screened for reactivity with antisera using Western blotting. Antibodies were detected against OppA, PstS and YrbD in serum from rabbits previously immunized with killed Y. pestis whole cells (see FIG. 1A), and antibodies against PiuA were detected in serum from humans who had recovered from plague (see FIG. 1B). Subsequently, the OppA, PstS, YrbD and PiuA proteins were selected for further evaluation as candidate protective antigens against Y. pestis challenge in a mouse model.

### Quantification of Antibody Response to Selected Proteins

Enzyme-linked immunosorbent assays (ELISA) were used to quantify the antibody responses against individual proteins in immunized mice. Each individual protein sample (˜50 μg) was applied to 48 wells of a 96 well plate. Serum from the test mice (n=6) was added at 1:500, 1:1000, 1:2000, 1:4000, 1:8000, 1:16000, 1:32,000, 1:64,000 dilutions. Bound antibodies in the serum samples were detected using HRP-conjugated anti-mouse IgG (1:10,000) or, where relevant, the individual IgG sub-classes (IgG1, IgG2a, IgG2b or IgG3; 1:2000), as the secondary antibody. 1,2-phenylethidiamine dihydrochloride and hydrogen peroxide were added as the substrate and the plates were incubated at room temperature for 10 min. Endpoint antibody titres were expressed as the maximum dilution of sample giving an absorbance of greater than 0.1 A_{490 nm} unit after subtraction of the absorbance due to nonspecific binding measured using negative control sera. Alternatively, IgG concentrations were estimated using His-tagged OppA as a standard where the mean±standard error values for each test point were calculated from ELISA data using the results from 4 mice after excluding the highest and lowest values from the data.

### Immunization and Protection Experiments.

Purified His-tagged proteins (OppA, PstS, YrbD and PiuA) were evaluated as candidate antigens in mouse immunization experiments. The proteins were prepared for immunization at 100 μg/ml concentrations in PBS and one of the following adjuvants: Alhydrogel (25% v/v) Alhydrogel (2%) (Superfos Biosector a/s. Vedback Denmark)), MPL+TDM adjuvant (50% v/v) MPL+TDM (Sigma-Aldrich Co. Ltd., Poole, UK)), Freunds Incomplete Adjuvant (1:1 protein:adjuvant (Sigma-Aldrich Co. Ltd, Poole, UK). 6 female Balb/c mice per group (8-12 weeks old) were used for all immunization experiments. Mice were administered 10 μg of each protein preparation by intramuscular injection (for Alhydrogel or MPL+TDM adjuvanted proteins) or intraperitoneal injection (for Freunds Incomplete adjuvanted proteins) on days 0, 14 and 28. Sera were collected from mice by retro-orbital bleeding on day 40. On day 57 mice were subcutaneously challenged with approximately 25 cfu of Y. pestis GB (known to have a median lethal dose of approximately 1 cfu in Balb/c mice by the subcutaneous route and then mice were observed daily until 15 days after challenge. One-way ANOVA with Tukey's multiple comparison post analysis test and statistical analysis of survival using the Mantel-Haenszel Logrank test were performed using GraphPad Prism version 3.02 for Windows (GraphPad Software, San Diego Calif.)

Groups of 6 mice were immunized with 3 doses of the individual proteins together with alhydrogel. Approximately 4 weeks after the final immunizing dose sera were taken and analysed for antibodies and the mice were challenged subcutaneously with 25 cfu (approximately 25 LD₅₀ doses) of a virulent strain of Y. pestis and monitored for survival. IgG subclass antibodies were present in pooled serum samples of animals immunized with PiuA, YrbD or OppA (endpoint titres of 32,000). In contrast, the IgG response to PstS was very low with an endpoint titre of 1,000.

The mean time to death of mice immunized against PstS, YrbD or PiuA was similar to control mice given alhydrogel only (Table 2). In contrast, mice immunized with OppA+alhydrogel showed a significant increase in time to death (p<0.05) compared to näive controls (Table 2). In a repeat experiment, in which mice were immunized with OppA+alhydrogel, the titre of serum IgG antibody in individual mice prior to challenge was measured. The results showed that the time to death correlated with the level of IgG to OppA, with the highest titres corresponding to the longest survival times (Table 3). In this experiment one of the immunized mice was alive at the termination of the experiment (15 days after challenge). This demonstrates that mice immunized with OppA show an increased time to death i.e. that OppA is protective.

TABLE 2
Mean times to death (MTTD) of mice which had
been immunised with PiuA, PstS, Oppa or YrbD
and challenged with 25 cfu of Y. pestis.

| Protein | MTTD (days) | P value vs naïve |
|---|---|---|
| PstS | 5.0 | P = 0.71 |
| YrbD | 5.5 | P = 0.86 |
| OppA | 8.2 | P = 0.03 |
| PiuA | 7.3 | P = 0.07 |
| Naïve | 5.2 | |

TABLE 3
Relationship of IgG responses to OppA in immunized mice and
survival following challenge with 25 cfu of Y. pestis.

| | | |
|---|---|---|
| Mouse | Time to death | IgG endpoint titre |

| | | | |
|---|---|---|---|
| 1 | 3 | days | 4000 |
| 2 | 4 | days | 8000 |
| 3 | 4 | days | 8000 |
| 4 | 6 | days | 8000 |
| 5 | 11 | days | 32000 |
| 6 | 15 | days (survivor) | 32000 |

Influence of Adjuvant on Antibody Response and Protection with OppA

To determine whether alternative adjuvants might enhance the protective response, mice were immunized with OppA adjuvanted with MPL+TDM or with Freunds Incomplete. When these mice were challenged with Y. pestis challenge there was no statistically significant increase in time to death compared with naïve mice.

The OppA-specific total IgG, IgG1, IgG2a, IgG2b and IgG3 antibody responses in the sera from these OppA-immunized mice were measured using ELISAs. These responses were compared with the responses in the mice which had been immunised with OppA+alhydrogel. (Table 4). Sera from naïve mice, which did not receive OppA or adjuvant, did not contain measurable levels of antibody to IgG2a, IgG2b or IgG3. However, a low level signal was obtained using the IgG1 ELISA (0.03 μg/ml). The mice given OppA in MPL+TDM developed low levels of all subclasses of IgG antibody. In comparison, mice given OppA in Alhydrogel or Freunds Incomplete adjuvants developed IgG antibodies. The level of IgG₁ antibody in mice immunized with OppA in Alhydrogel (2.1±0.3 mg/ml) was approximately 1.5 times higher than level of IgG₁ antibody in mice given OppA in Freunds Incomplete adjuvant (1.0±0.2 mg/ml).

TABLE 4
Sub-classes of IgG raised against Y. pestis OppA using different adjuvants

| Antibody subclass | Alhydrogel | | MPL + TDM | | Freunds Incomplete | |
|---|---|---|---|---|---|---|
| IgG1 (μg/ml) | 2.1 | (8.7 × 10⁻²)† | 0.12 | (5.5 × 10⁻³) | 1.1 | (6.2 × 10⁻²) |
| IgG2a (μg/ml) | 0.43 | (4.1 × 10⁻²) | 2.5 × 10 − 2 | (1.4 × 10⁻³) | 0.95 | (3.2 × 10⁻²) |
| IgG2b (μg/ml) | 0.36 | (8.8 × 10⁻²) | 1.5 × 10 − 2 | (2.5 × 10⁻³) | 0.37 | (4.6 × 10⁻²) |
| IgG3 (μg/ml) | 1.2 | (0.15) | 3.0 × 10 − 3 | (0) | 1.67 | (0.15) |

†Numbers in parentheses refer to the standard error deviation.

Crystallization of OppA—Crystals of OppA were obtained from both 96-well sitting drop and 24-well hanging drop vapor diffusion methods. The optimized crystals were cryo-protected in liquid nitrogen prior to data collection. The OppA data set was collected at ESRF (the European Synchrotron Radiation Facility) beamline ID29, with an ADSC Quantum 4 CCD detector. All data were collected from frozen crystals at 100 K and processed using Denzo/Scalepack for further analysis.

The Y. pestis OppA structure was solved by the molecular replacement method, using S. typhimurium OppA as a starting model in Phaser as implemented in the CCP4 suite. The density modification and B-factor refinement were carried out by ARP/wARP and CNS followed by manual model building in Program O. All structural figures were prepared using Pymol.

The structure of OppA was solved at 2.0 Å in order to obtain more information on the likely antigenic regions of the protein. The refinement statistics are shown in Table 5. Refinement of the OppA structure decreased both the R-factor and the R-free value to R=21.2% and R_{free}=23.9%. OppA was crystallized and the structure was solved with bound substrate (tri-lysine), demonstrating an identical function to the homologous S. typhimurium OppA protein. The overall crystal structure of OppA with tri-lysine reveals that the ligand tri-peptide is completely enclosed in the protein interior, a mode of binding that normally imposes tight specificity (FIG. 2). The protein fulfills the hydrogen bonding requirements and accommodates the peptide side chains in voluminous hydrated cavities. The high resolution structure of OppA from S. typhimurium with various peptide ligands has previously been solved. The OppA proteins from S. typhimurium and Y. pestis are very similar sharing 79% sequence homology and 85% sequence identity. In addition, the two structures can be superimposed with a root mean square deviation of 0.49% (for 517 Cα atoms). Both structures bind tri-lysine at the same position, and the B-factor for the binding pocket region is low. In terms of amino acid sequence, three regions (residues 50-60, 126-134, 456-466) are far less conserved, but all these regions are located at the beginning or end of a helix, and there is no unique motif or significant conformational differences between the two structures.

TABLE 5
OppA refinement statistics
Refinement

| | |
|---|---|
| Resolution range (Å) | 30-2.0 |
| No. of molecule in asymmetric unit | 1 |
| No. of protein atom | 517 |
| No. of water molecules | 414 |
| R_{work}† (%) | 21.0 (35.8)* |
| R_{free}¶ (%) | 23.9 (38.0)* |
| R.m.s.d bond length (Å) | 0.049 |
| R.m.s.d bond angles (°) | 2.68 |

*The numbers in the parentheses correspond to the highest resolution shell.
†R_{work} = Σ∥Fo| − |Fc∥/Σ|Fo|
¶R_{free} is the value for R-value for a subset of 5% of randomly selected reflection data, which were excluded from refinement.

### Results Summary

High quality Y. pestis ABC transporter proteins in the quantities required for both immunological and structural studies were cloned expressed and purified. Initially, OppA, the periplasmic oligopeptide-binding protein, PstS, a periplasmic phosphate-binding protein, and YrbD, a putative toluene transport protein were immuno-reactive with the rabbit antisera, indicating that these proteins were expressed by Y. pestis when cultured in vitro. PiuA, an outer membrane iron uptake channel was immuno-reactive with convalescent human sera indicating that this protein is recognised by the immune system during infection. The finding that PiuA was not protective in our model against Y. pestis infection is interesting since it has been shown that PiuA in Streptococcus pneumoniae is surface located and that recombinant PiuA protein is able to provide protection against systemic S. pneumoniae infection (Brown, J. S. et al, Infect. Immun. 69 (2001) pp 6702-6706).

The results from the in vivo challenge experiments showed that mice immunized with OppA showed a significant increase in survival rate compared to controls. The correlation between serum IgG titres measured for the individual animals and survival following challenge suggests that antibody to OppA was responsible for protection. In addition, the protective effect of OppA seems to be adjuvant specific. Co-administration of OppA+Freunds Incomplete adjuvant produced an immune response but no increase in time to death compared to controls. The IgG subclass profile stimulated by OppA given with Freunds Incomplete adjuvant differed from that of OppA delivered with Alhydrogel, perhaps reflecting the difference in protection afforded. OppA and Alhydrogel stimulated the highest titres of OppA-specific IgG1. Since studies on the F1 and V antigens have suggested an important role for the IgG1 subclass in protection against Y. pestis infection, the difference in OppA-specific IgG1 levels may reflect the difference in protection afforded by OppA in the various adjuvants.

OppA is a component of the oligopeptide (Opp) ABC transporter, binding substrate and delivering it to the membrane bound complex OppBCDF for ATP-mediated transport across the membrane. Y. pestis is predominantly an extracellular pathogen, which would allow antibody to bind to bacterial cells. However, OppA is generally considered to be located in the periplasm, a region of the bacterial cell with limited accessibility to the host immune system. The data presented herein suggests that OppA is available to the immune system. It is possible that OppA-specific antibody could directly block the oligopeptide uptake system. Alternatively, it may have an opsonising effect, promoting bacterial uptake into professional antigen presenting cells.

The high level of sequence and structural homology between the OppA from Y. pestis and the OppA from S. typhimurium indicate the highly conserved nature of the proteins. The major differences between the two proteins are localized to small regions of exposed loops (FIG. 2A), regions of the protein most favourable to immune system recognition. However there is no evidence of immunological cross reactivity between the OppA from Y. pestis and the OppA from S. typhimurium.

Sequence ID no.1: Y. pestis OppA
atgaccaacatcacaaagaagaatctcattacagctgctgtagccgttgc
aatgagcatgatggctgcgggaaatacgtttgctgccaatgttcccacag
gcgtacaactggctgagaagcaagtgttagttcgtaataatggctccgag
cctcaatcgttggatccacataaaattgaaggggtacctgaatccaatat
ttcacgtgatctacttgaagggctggtgattaatgaccctaatggcaata
ttgtaccgggagccgcagagagttgggataataaagactttaaagtctgg
acgtttaatattcgtaaagatgccaaatggtcaaatggtgaccctgtaac
ggcacaggattttgtttacagttggcaacgtctggctgatcctaaaacag
tctctccttatgcaagctatttgcaatacgctcatctcactaatatcgat
gacattattaccggtaaagccgcgcctgacacgttgggtgtaaaagcgtt
ggatgatcatacgctggaagtgacattgtctgagcctgttccttatttgg
ataagctgttagctcacccgtcaatgtcaccggtgaataaaaccgtggtc
gagaaatttggtgaaaaatggacccaaccacaaaattttgtggggaatgg
tgcttataaactaaaagattggattgttaacgaacgtatcgtattagaac
gcagtcctacgtattgggataatgcgaaaacagtcatcaatcaggtgact
tatttgccaatttcttctgaagtgacggatgttaaccgctatcgcagcgg
cgaaattgatatgacgtataacaacatgccaattgaattattccaaaaac
tgaaaaaagaaattccagatcaagtccatgttgatccctatctgtgtact
tattactacgaaattaacaatcaaaaagccccttttacggatgctcgtgt
acgtgaagcgctgaaattgggtatggaccgcgatatcatcgtcaataaag
ttaaaaatcagggagatttacccgcttatggtttcaccccaccctatacc
agtggcgcggagctgacgccgccagagtggtttagctggacacaagaaaa
acgtaatgaagtagcgaaaaaactattggctgaagccgggtacactaaag
acaatccactgaagtttagcttgttatataacacctccgatttacataaa
aaattagccattgctgccgcctctatttggaagaaaaatctgggtgtaga
cgttaaactggaaaaccaagagtggaaaacgttcctggatacccgtcacc
aggggacttatgatgttgcccgtgccgcttggtgtgccgattacaatgag
ccatcctccttcctaaatatgatgttatctaacagtagcaacaacaccac
ccattataaaagttcggtatttgacaagctgatagaagacacgttgaaag
tgaaatcggaaaaagagcgtgctgacttgtatcaacaagcggaaatccag
ttggataaagattctgccattgtgcccgtgttctattacgtgagtgcgcg
tttagtgaaaccttatgttggcggctacacgggtaaggatccactggaca
atatgcacgttaaagatctttatattatcaagcagtaa
Sequence ID no.2: (AA sequence):
QLAEKQVLVRNNGSEPQSLDPHKIEGVPESNISRDLLEGLVINDPNGNIV
PGAAESWDNKDFKVWTFNIRKDAKWSNGDPVTAQDFVYSWQRLADPKTVS
PYASYLQYAHLTNIDDIITGKAAPDTLGVKALDDHTLEVTLSEPVPYLDK
LLAHPSMSPVNKTVVEKFGEKWTQPQNFVGNGAYKLKDWIVNERIVLERS
PTYWDNAKTVINQVTYLPISSEVTDVNRYRSGEIDMTYNNMPIELFQKLK
KEIPDQVHVDPYLCTYYYEINNQKAPFTDARVREALKLGMDRDIIVNKVK
NQGDLPAYGFTPPYTSGAELTPPEWFSWTQEKRNEVAKKLLAEAGYTKDN
PLKFSLLYNTSDLHKKLAIAAASIWKKNLGVDVKLENQEWKTFLDTRHQG
TYDVARAAWCADYNEPSSFLNMMLSNSSNNTTHYKSSVFDKLIEDTLKVK
SEKERADLYQQAEIQLDKDSAIVPVFYYVSARLVKPYVGGYTGKDPLDNM
HVKDLYIIKQ

1. A polypeptide derived from the periplasmic space of Yersinia pestis or a variant of said polypeptide or a fragment of any of these wherein the polypeptide, variant or fragment produces a protective immune response against Y. pestis, for use when administered to a human or animal.

2. The polypeptide of claim 1 wherein the polypeptide is a periplasmic solute binding protein, or a variant of said polypeptide or a fragment of any of these that produces a protective immune response against Y. pestis when administered to a human or animal.

3. The polypeptide claim 1 wherein the polypeptide has the amino acid sequence of SEQ ID NO:2, or is a variant thereof or is a fragment of any of these that produces a protective immune response against Y. pestis when administered to a human or animal.

4. A pharmaceutical composition comprising a polypeptide derived from the periplasmic space of Yersinia pestis or a variant of said polypeptide that produces a protective immune response against Y. pestis when administered to a human or animal, or a fragment of any of these that produces a protective immune response against Y. pestis when administered to a human or animal, in combination with a pharmaceutically acceptable carrier or excipient.

5. The pharmaceutical composition of claim 4 wherein the composition further comprises an adjuvant.

6. The pharmaceutical composition of claim 4 wherein the composition further comprises an additional immunogenic polypeptide derived from Yersinia pestis.

7. The pharmaceutical composition of claim 4 wherein the additional immunogenic polypeptide is a protective antigen for Yersinia pestis.

8. The pharmaceutical composition of claim 5 wherein the protective antigen is selected from the group consisting of the Y. pestis V antigen, the Y. pestis F1 antigen and a fusion protein comprising both the Y. pestis V and F1 antigens.

9. A nucleic acid molecule that encodes the polypeptide of claim 1.

10. The nucleic acid molecule of claim 9 wherein the polypeptide has the amino acid sequence of SEQ ID NO:2.

11. The nucleic acid molecule of claim 9 wherein the nucleic acid molecule has the nucleic acid sequence of SEQ ID NO:1.

12. A pharmaceutical composition comprising the nucleic acid molecule of claim 9 in combination with a pharmaceutically acceptable carrier.

13-14. (canceled)

15. An antibody raised against the polypeptide of claim 1.

16. A method of protecting a human or animal body from the effects of infection with Yersinia pestis comprising administering to the body the pharmaceutical composition of claim 4.

17. A method of protecting a human or animal body from the effects of infection with Yersinia pestis comprising administering to the body the pharmaceutical composition of claim 12.


SEQUENCE LISTING (4 sequences)

SEQ ID NO: 1 - DNA, 1638 bp, Yersinia pestis
atgaccaacatcacaaagaagaatctcattacagctgctgtagccgttgcaatgagcatgatggctgcgggaaatacgtttgctgccaatgttcccacaggcgtacaactggctgagaagcaagtgttagttcgtaataatggctccgagcctcaatcgttggatccacataaaattgaaggggtacctgaatccaatatttcacgtgatctacttgaagggctggtgattaatgaccctaatggcaatattgtaccgggagccgcagagagttgggataataaagactttaaagtctggacgtttaatattcgtaaagatgccaaatggtcaaatggtgaccctgtaacggcacaggattttgtttacagttggcaacgtctggctgatcctaaaacagtctctccttatgcaagctatttgcaatacgctcatctcactaatatcgatgacattattaccggtaaagccgcgcctgacacgttgggtgtaaaagcgttggatgatcatacgctggaagtgacattgtctgagcctgttccttatttggataagctgttagctcacccgtcaatgtcaccggtgaataaaaccgtggtcgagaaatttggtgaaaaatggacccaaccacaaaattttgtggggaatggtgcttataaactaaaagattggattgttaacgaacgtatcgtattagaacgcagtcctacgtattgggataatgcgaaaacagtcatcaatcaggtgacttatttgccaatttcttctgaagtgacggatgttaaccgctatcgcagcggcgaaattgatatgacgtataacaacatgccaattgaattattccaaaaactgaaaaaagaaattccagatcaagtccatgttgatccctatctgtgtacttattactacgaaattaacaatcaaaaagccccttttacggatgctcgtgtacgtgaagcgctgaaattgggtatggaccgcgatatcatcgtcaataaagttaaaaatcagggagatttacccgcttatggtttcaccccaccctataccagtggcgcggagctgacgccgccagagtggtttagctggacacaagaaaaacgtaatgaagtagcgaaaaaactattggctgaagccgggtacactaaagacaatccactgaagtttagcttgttatataacacctccgatttacataaaaaattagccattgctgccgcctctatttggaagaaaaatctgggtgtagacgttaaactggaaaaccaagagtggaaaacgttcctggatacccgtcaccaggggacttatgatgttgcccgtgccgcttggtgtgccgattacaatgagccatcctccttcctaaatatgatgttatctaacagtagcaacaacaccacccattataaaagttcggtatttgacaagctgatagaagacacgttgaaagtgaaatcggaaaaagagcgtgctgacttgtatcaacaagcggaaatccagttggataaagattctgccattgtgcccgtgttctattacgtgagtgcgcgtttagtgaaaccttatgttggcggctacacgggtaaggatccactggacaatatgcacgttaaagatctttatattatcaagcagtaa

SEQ ID NO: 2 - protein, 510 aa, Yersinia pestis
QLAEKQVLVRNNGSEPQSLDPHKIEGVPESNISRDLLEGLVINDPNGNIVPGAAESWDNKDFKVWTFNIRKDAKWSNGDPVTAQDFVYSWQRLADPKTVSPYASYLQYAHLTNIDDIITGKAAPDTLGVKALDDHTLEVTLSEPVPYLDKLLAHPSMSPVNKTVVEKFGEKWTQPQNFVGNGAYKLKDWIVNERIVLERSPTYWDNAKTVINQVTYLPISSEVTDVNRYRSGEIDMTYNNMPIELFQKLKKEIPDQVHVDPYLCTYYYEINNQKAPFTDARVREALKLGMDRDIIVNKVKNQGDLPAYGFTPPYTSGAELTPPEWFSWTQEKRNEVAKKLLAEAGYTKDNPLKFSLLYNTSDLHKKLAIAAASIWKKNLGVDVKLENQEWKTFLDTRHQGTYDVARAAWCADYNEPSSFLNMMLSNSSNNTTHYKSSVFDKLIEDTLKVKSEKERADLYQQAEIQLDKDSAIVPVFYYVSARLVKPYVGGYTGKDPLDNMHVKDLYIIKQ

SEQ ID NO: 3 - DNA, 33 bp, Artificial sequence
  Synthetic sequence Primer
gaattcatgaccaacatcacaaagaagaatctc

SEQ ID NO: 4 - DNA, 36 bp, Artificial sequence
  Synthetic sequence Primer
ctcgagctgcttgataatataaagatctttaacgtg